(19) United States
(12) Patent Application Publication
(10) Pub. No.: US 2021/0353588 A1
(43) Pub. Date: Nov. 18, 2021

(54) DIETARY SUPPLEMENT COMPOSITION COMPRISING OF CANNABIS SATIVA EXTRACTS

(71) Applicant: VICTOR PALINCZAR, Trenton, NJ (US)

(72) Inventor: VICTOR PALINCZAR, Trenton, NJ (US)

(21) Appl. No.: 16/873,582

(22) Filed: May 12, 2020

(51) Int. Cl. A61K 31/352; A61K 31/05; A61K 47/44; A61K 31/355; A61K 47/22; A23L 33/105; A23L 33/115; A23L 33/15

(52) U.S. Cl. CPC A61K 31/352; A61K 31/05; A61K 47/44; A61K 31/355; A23V 2002/00; A23L 33/105; A23L 33/115; A23L 33/15; A61K 47/22

(57) ABSTRACT

Dietary supplement compositions for relief of human and animal ailments comprising from 0.1 percent to 50 percent of a Cannabis sativa extract or combinations of a Cannabis sativa extracts and from 10 percent to 99.9 percent of a naturally derived edible oil or combinations of a naturally derived edible oils, where the naturally derived edible oil has a total saturated fat content of less than about 15 percent and a total polyunsaturated fat content greater than 44 percent, said edible oil or combination of said edible oils enhance sublingual and buccal pouch absorption of the Cannabis sativa extract and improve the overall taste of the composition. The composition's taste may be further enhanced with the addition of compatible flavoring agents. To improve the absorption of the Cannabis sativa extract and the compatibility of said flavoring agents, surface active agents may be incorporated. The composition may still further be enhanced by addition of various antioxidants and preservatives.

### CROSS-REFERENCE TO RELATED APPLICATIONS

This patent application is a continuation of Provisional Patent Application No. 62/920,717

### FEDERALLY SPONSORED RESEARCH OR DEVELOPMENT

None

### FIELD OF INVENTION

What has been discovered are unique and novel, essentially anhydrous, dietary supplement compositions comprising: a) a Cannabis sativa extract and b) naturally derived edible oil that improves the bioavailability of said extract that relieve joint pain, anxiety and other naturally occurring ailment. The compositions may additionally comprise of various compatible surface active agents, vitamins, antioxidants, flavors, and preservatives.

### BACKGROUND OF INVENTION

Dietary supplement compositions and more specifically, Dietary supplement compositions that contain Cannabis sativa extract that are currently marketed contain edible oils high in saturated fats and low in polyunsaturated fats. These oils leave a residual film in the mouth and lessen the degree of sublingually and/or buccal absorption of the Cannabis sativa extract; more specifically, Cannabidiol, also know as CBD and a major component of the Cannabis sativa extract. Decreasing the absorption into the blood plasma prevents the CBD from performing to its fullest ability. In compositions containing high level of saturated edible oil the CBD is unabsorbed, swallowed and becomes metabolized.

Another disadvantage of currently marketed Cannabis sativa extract products is that the taste in combination with the CBD component is less then desirable; the mouth is left with a mild rancid sensation. Additionally, due to the high levels of saturated fat content, precipitates of long chain molecules form, causing a non-homogeneous mixture with a potential to deliver disproportions of CBD concentrations. Mixing the product by hand is required prior to use; the problem is exacerbated if the product is stored in a cool or cold location. Preservation of such compositions, however, are enhanced in cold storage; the use of oils containing high levels of saturated fats becomes problematic.

The use of Cannabis sativa extracts and more specifically Cannabinoid compounds have been cited for pain relief. Wallace, in U.S. Patent describes Cannabinoid compositions relieving analgesia and reducing inflammation by delivery topical liniment composition. Stinchcomb, in U.S. Pat. No. 8,293,786—Alltranz Inc. describes the use of Cannabidiol (CBD) for use in transdermal applications. Lewis, in U.S. Pat. No. 9,095,554—Biotect Institute, describes Cannabinoid compositions in combination with terpene oils. The present invention is to be taken orally. Murty, et al in U.S. Pat. No. 9,265,724 describes the use of Cannabinoid compositions in combination with triglycerides and glycerides. The present invention is free of triglycerides and glycerides. In non-continuous U.S. Pat. Nos. 9,949,9936 to 10,137,095 issued to Guy—G W Pharma, describe the use of Cannabinoid compounds an more specifically compositions comprised of CBD as a method for the treatment of epilepsy using sesame oil as a carrier at levels of eighty percent or greater. The use of sesame oil in the present invention serves only as a flavoring agent for domestic animal compositions at a use level of two percent or less. Whittle in U.S. Pat. No. 6,730,330 G W Pharma describes the use of tetrahydrocannabinol (THC) and cannabidiol (CBD) in Pharmaceutical formulations that make use of a matrix comprising of at least one agent which is both a self-emulsifier and a cannabinoid solubilizer. In the present invention a self-emulsifier and a cannabinoid solubilizer is not employed. Additionally, Whittle in U.S. Pat. No. 6,730,330 G W Pharma makes broad and blanket claims for the use of numerous surface active agents that range in Hydrophilic/Lipophilic balance from 1 to about 30; they do not scientifically corroborate their functionally within the patent. The present invention is an improvement over Whittle, it specifically employs surface active agents limited to non-ionic surface active agents having a Hydrophilic Lipophilic balance greater than 9 and less than 25. Additionally, Whittle fails to describe the safety of the surface active agents used for oral consumption; his use of a broad spectrum of fatty acids, act as carriers. In the present invention, only FDA food grade surface active agents are used in combination with polyunsaturated acids, high in alpha linolenic acid, are employed.

### SUMMARY OF THE INVENTION AND DETAILED DESCRIPTION

The present invention is to provide an improved means to relieve various ailments such as joint pain and anxiety associated with domestic animals and humans by increasing degree of bioavailability through sublingually and/or buccal absorption of the composition, and more specifically, increasing degree of bioavailability through sublingually and/or buccal absorption of Cannabidiol henceforth referred to CBD; a component of Cannabis sativa. The short-comings of the currently marketed over-the-counter CBD products have been greatly decreased by the present invention described herein.

What has been discovered is that when CBD, is combined with an edible oil or a combination of edible oils having a saturated fat content of about less than about 15 percent by weight and having a polyunsaturated fat content greater than 44 percent by weight, allow for a more rapid sublingual and/or buccal absorption of CBD; a unique and novel composition results that allows for greater efficacy and efficiently of the CBD compound.

The attributes of CBD use for the relief of pain and anxiety is well documented in the literature. In a recent study conducted by the Kogan, et al, indicated that over eighty percent of veterinarians who participated in a use study, evaluating the use of CBD products, perceived that CBD was very helpful or somewhat helpful for conditions of acute pain, chronic pain and anxiety in dogs.

Another objective of the present invention is to provide an improved means to relieve various medical ailments such as depression, and nervous disorders such as tourette syndrome associated with humans by increasing the degree of bioavailability through sublingually and/or buccal absorption of the composition, and more specifically, increasing the degree of bioavailability through sublingually and/or buccal absorption of Delta-9-tetrahydrocannabinol also known as Δ-9-THC and it's isomers that are components of Cannabis sativa. The medicinal properties of these compounds are well documented as described in a book written by Michael Backes, and Andrew Weil, MD, entitled: “Cannabis Pharmacy: The Practical Guide to Medical Marijuana.” Currently there are no known medicinal products marketed that are used for sublingually and/or buccal absorption of Delta-9-tetrahydrocannabinol. The present invention described herein is advantageous over other forms of dosages containing Delta-9-tetrahydrocannabinol. What has been discovered is that when Delta-9-tetrahydrocannabinol, is combined with an edible oil or a combination of edible oils having a saturated fat content of about less than about 15 percent and a polyunsaturated fat content greater than 44 percent by weight, allow for a more rapid sublingual and/or buccal absorption of Delta-9-tetrahydrocannabinol a unique and novel composition results that allows for greater efficacy and efficiently of the compound.

Another objective of the present invention is the incorporation of high levels of alpha linolenic acid and gamma linolenic acid that are inherent to the polyunsaturated edible oils used in the present invention. Compositions of the present may contain up to fifty-five percent by weight of alpha linolenic acid. The nutritional attributes of alpha linolenic acid are well documented in the literature and are recognized by the U.S. Food and Drug Administration, hereinafter referred to as the FDA, as a nutritional ingredient. Compositions that meet the FDA requirements may be listed as dietary supplement products containing alpha linolenic acid as a “nutritional” ingredient. When alpha linolenic acid is use in the present invention, FDA approved claims such as: “Excellent source of ALA.” or “High in ALA” or “Rich in ALA” can be made.

Additional benefits of alpha linolenic acid, is further documented in a publication by The National Institute of Health dated Nov. 21, 2018, entitled: Omga-3 Fatty Acids, Fact Sheet for Health Professionals.

It has been discovered that in the present invention the combination of the attributes of alpha linolenic acid and the Cannabis sativa extract act synergistically, to alleviate pain, joint stiffness and anxiety.

It has also been discovered that when Cannabis sativa extract, is combined with edible oils comprising of low saturated fat levels, and more specifically, edible oils comprising of less than 15 percent by weight of saturated fats and greater than 44 percent by weight of polyunsaturated fats, are used in compositions of this invention, the oily residual feeling in the mouth is practically eliminated.

It has further been discovered that when Cannabis sativa extract, is combined with edible oils comprising of low saturated fat levels, and more specifically, edible oils comprising of less than 15 percent by weigh of saturated fats and greater than 44 percent by weight of polyunsaturated fats are used in compositions of this invention, eliminate any taste of rancidity.

It has also further been discovered that when Cannabis sativa extract, is combined with edible oils comprising of low saturated fat levels, and more specifically, edible oils comprising of less than 15 percent by weight of saturated fats and greater than 44 percent weight of polyunsaturated fats are used in compositions of this invention, eliminate precipitation of solids and the necessity to manually mix the composition.

It has still further been discovered that when Cannabis sativa extract, is combined with edible oils comprising of low saturated fat levels, and more specifically, edible oils comprising of less than 15 percent by weigh of saturated fats and greater than 44 percent by weight of polyunsaturated fats are used in compositions of this invention, allow the compositions to be stored in a cold environment thus extending the shelf-life of the compositions.

It has still further been discovered that when Cannabis sativa extract, is combined with edible oils comprising of low saturated fat levels, and more specifically, edible oils comprising of less than 15 percent by weight of saturated fats and greater than 44 percent by weight of polyunsaturated fats are used in compositions of this invention, in combination with Sesame Oil, makes the taste of these compositions highly desirable for canine use.

It has still further been discovered that when Cannabis sativa extract, is combined with edible oils comprising of low saturated fat levels, and more specifically, edible oils comprising of less than 15 percent by weight of saturated fats and greater than 44 percent by weight of polyunsaturated fats are used in compositions of this invention, in combination with Sesame Oil, makes the taste of these compositions highly desirable for feline use.

It has still further been discovered that when Cannabis sativa extract, is combined with edible oils comprising of low saturated fat levels, and more specifically, edible oils comprising of less than 15 percent by weight of saturated fats and greater than 44 percent by weight of polyunsaturated fats are used in compositions of this invention, in combination with Sesame Oil, makes the taste of these compositions highly desirable for equine use especially equines that need to be tamed and/or those bred for racing.

It has still further been discovered that when Cannabis sativa extract, is combined with edible oils comprising of low saturated fat levels, and more specifically, edible oils comprising of less than 15 percent by weight of saturated fats and greater than 44 percent by weight of polyunsaturated fats are used in compositions of this invention, make these compositions highly desirable for human use to decrease joint pain, increase awareness and improve libido.

It has still further been discovered that when Cannabis sativa extracts used in this invention comprise of compounds other then CBD, and are combined with the edible oils of this invention, they may serve as medical compositions to enhance the delivery of one or more of the following compounds: Cannabinol also known as CBN; Cannabigerol also known as CBG; Cannabidiolic acid also known as CBD-A; Cannabichromene also known as CBC; Delta-9-tetrahydrocannabinol also known as Δ-9-THC; Delta-9-tetrahydrocannabinol Acid also known as Δ-9-THC-A; Delta-8-tetrahydrocannabinol also known as Δ-8-THC; and Tetrahydrocannabivarin also known as THC-V;

It has still further been discovered that an additional benefit of the edible oils, comprising of high levels of polyunsaturated fats, used in compositions of the present invention allow for greater dissolution of the vitamins and antioxidants described herein.

The edible oils comprising of low saturated fat levels, and more specifically, edible oils comprising of less than 15 percent by weight of saturated fats and greater than 44 percent by weight of polyunsaturated fats comprises, but not limited to: Borage Seed Oil, Chia Seed Oil, Cranberry Seed Oil, Elderberry Seed Oil, Flax Seed Oil, Grape Seed Oil, Hemp Seed Oil, Maracuja Seed Oil, Perilla Seed Oil, Poppy Seed Oil, Safflower Seed Oil, Soya Bean Oil and Sunflower Seed Oil.

The surface active agents comprise, but not limited to: of one or more of FDA approved food grade non-ionic surfactants having a hydrophilic/lipophilic (HLB) balance greater than 9 and less 25. Surfactants such as: Tween 85 (Polyoxyethylene sorbitan trioleate), Tween 81 (Polyoxyethylene sorbitan monooleate), Tween 80 (Polyoxyethylene sorbitan monooleate), Tween 65 (Polyoxyethylene sorbitan tristearate), Tween 61 (Polyoxyethylene sorbitan monostearate), Tween 60 (Polyoxyethylene sorbitan monostearate), Tween 40 (Polyoxyethylene sorbitan monopalmitate), Tween 21 (Polyoxyethylene sorbitan monolaurate), Tween 20 (Polyoxyethylene) sorbitan monolaurate; non-ionic surfactants in the form of ethers such as: Brij 020 (Polyoxyethylene (20) oleyl ether), Brij L4 (Polyoxyethylene (4) lauryl ether), Brij S100 (Polyoxyethylene (100) stearyl ether); and non-ionic surfactants in the form Copolymers of polyethylene glycols and polypropylene glycols such as: Pluronic F68 and F127.

The vitamins comprise of but not limited to: one or a combination of the following; Vitamins such as: Vitamin A (retinol), Vitamin C as Ascorbyl Palmitate, Vitamin D₃, Vitamin E including tocotrienol and tocopherol and its esters.

The antioxidants comprise of but not limited to: one or a combination of the following: Alpha Lipoic Acid, Carotenoid terpenoids such as: Alpha-carotene, Astaxanthin, Beta-carotene, Canthaxanthin, Cryptoxanthin, Lutein, Lycopene, and Zeaxanthin; Flavonoids such as: Flavones such as: Apigenin, Luteolin, and Tangeritin; Flavonols such as: Isorhamnetin, Kaempferol, Myricetin, and Quercetin; Flavanones such as: Eriodictyol, Hesperetin, and Naringenin; Flavanols and their polymers such as: Catechin, gallocatechin and their corresponding gallate esters, Epicatechin, epigallocatechin and their corresponding gallate esters, and Theaflavin and its gallate esters; Isoflavone phytoestrogens such as: Daidzein, Genistein, and Glycitein; Stilbenoids such as: Resveratrol, and Pterostilbene; Anthocyanins: Cyanidin, Delphinidin, Malvidin, Pelargonidin, Peonidin, and Petunidin; Phenolics such as: Curcumin, Flavonolignans, Xanthones, and Eugenol.

The flavoring agents comprise of sesame oil and other compatible flavoring agents known to those in the arts.

The preservatives comprise of one or a combination of the following: Butylated Hydroxy Toluene (BHT), and Butylated Hydroxy Anisole (BHA).

### EXAMPLES

### Example 1

### Canine Product

| Supplier | Ingredients | % W/W |
|---|---|---|
| PUREVINE PRODUCTS | CHIA SEED OIL | 48.30 |
| PUREVINE PRODUCTS | FLAX SEED OIL | 20.00 |
| PUREVINE PRODUCTS | BORAGE SEED OIL | 15.00 |
| GREEN BEAUTY | HEMP SEED OIL | 10.00 |
| CRAN NATURELLE | CRANBERRY SEED OIL | 4.00 |
| PUREVINE PRODUCTS | SESAME SEED OIL | 1.00 |
| GLOBAL CANNABINOIDS | Cannabidiol (CBD) | 1.00 |
| ORYZA CHEMICAL | ALPHA LIPOIC ACID | 0.50 |
| BASF | Tocopherol (Vitamin E) | 0.10 |
| BUTYLHYDROXYANISOLE | EASTMAN | 0.10 |
| TOTAL | | 100.00 |

### Example 2

### Feline Product

| Supplier | Ingredients | % W/W |
|---|---|---|
| PUREVINE PRODUCTS | CHIA SEED OIL | 49.30 |
| PUREVINE PRODUCTS | FLAX SEED OIL | 20.00 |
| PUREVINE PRODUCTS | BORAGE SEED OIL | 15.00 |
| GREEN BEAUTY | HEMP SEED OIL | 10.00 |
| CRAN NATURELLE | CRANBERRY SEED OIL | 4.00 |
| PUREVINE PRODUCTS | SESAME SEED OIL | 1.00 |
| GLOBAL CANNABINOIDS | Cannabidiol (CBD) | 0.50 |
| BASF | Tocopherol (Vitamin E) | 0.10 |
| BUTYLHYDROXYTOLUENE | EASTMAN | 0.10 |
| TOTAL | | 100.00 |

### Example 3

### Equine Product

| Supplier | Ingredients | % W/W |
|---|---|---|
| PUREVINE PRODUCTS | CHIA SEED OIL | 39.00 |
| PUREVINE PRODUCTS | FLAX SEED OIL | 20.00 |
| PUREVINE PRODUCTS | BORAGE SEED OIL | 15.00 |
| GREEN BEAUTY | HEMP SEED OIL | 10.00 |
| CRAN NATURELLE | CRANBERRY SEED OIL | 4.00 |
| PUREVINE PRODUCTS | SESAME SEED OIL | 1.00 |
| GLOBAL CANNABINOIDS | Cannabidiol (CBD) | 10.00 |
| BASF | Tocopherol (Vitamin E) | 1.00 |
| TOTAL | | 100.00 |

### Example 4

### Human Product

| Supplier | Ingredients | % W/W |
|---|---|---|
| PUREVINE PRODUCTS | GRAPE SEED OIL | 50.40 |
| PUREVINE PRODUCTS | SAFFLOWER SEED OIL | 20.00 |
| CRODA, INDUSTRIES | TWEEN 20 | 15.00 |
| CRAN NATURELLE | CRANBERRY SEED OIL | 5.00 |
| ELDER PURE | ELDERBERRY SEED OIL | 5.00 |
| GLOBAL CANNABINOIDS | Cannabidiol (CBD) | 4.00 |
| PARCHEM | EUGENOL | 0.50 |
| BASF | Tocopherol (Vitamin E) | 0.10 |
| TOTAL | | 100.00 |

### Example 5

### Medicinal Product

| Supplier | Ingredients | % W/W |
|---|---|---|
| PUREVINE PRODUCTS | GRAPE SEED OIL | 50.00 |
| PUREVINE PRODUCTS | SAFFLOWER SEED OIL | 20.00 |
| CRODA, INDUSTRIES | TWEEN 20 | 15.00 |
| CRAN NATURELLE | CRANBERRY SEED OIL | 5.00 |
| ELDER PURE | ELDERBERRY SEED OIL | 5.00 |
| N/A* | Tetrahydrocannabinol (THC) | 4.00 |
| BEST FLAVORS | SPEARMINT FLAVOR | 1.00 |
| BASF | Tocopherol (Vitamin E) | 0.10 |
| TOTAL | | 100.00 |

*Obtained by extraction from plants containing Cannabis sativa.

Procedure:
A) Charge all edible oils to a sanitized make vessel;
B) Charge the Cannabis extract, warm gently to about 40 degrees C. and mix until dissolution;
C) Charge the surface active agent, antioxidant, and preservative where each ingredient is called for, mix until dissolution;
D) Cool to 25 degrees C.;
E) Charge the Vitamin E, mix until dissolution;
F) Charge the FLAVOR when called for, mix until dissolution.
G) Filter the composition through a 5 micron filter.

### REFERENCES CITED

### U.S PATENT DOCUMENTS

| | | |
|---|---|---|
| 6,056,971 | May 2000 | Goldman |
| 6,184,255 | February 2001 | Mae, et al |
| 6,300,377 | October 2001 | Chopra |
| 6,730,330 | May 2004 | Whittle, et al |
| 6,740,338 | May 2004 | Chopra |
| 8,293,786 | October 2012 | Stinchcomb |
| 9,095,554 | May 2015 | Lewis |
| 9,265,724 | February 2016 | Murty, et al |
| 9,949,936 | April 2018 | Guy, et al |
| 10,137,095 | November 2018 | Guy, et al |

### OTHER PUBLICATIONS

Lancz et al., INTERACTION OF DELTA-9-TETRAHYDROCANNABINOL WIm HERPESVIRUSES AND CULTURAL CONDmONS ASSOCIATED WIm DRUG-INDUCED ANTI-CELLULAR EFFECTS, Departments of Medical Microbiology and Immunology, Anatomy, and Pharmacology and Therapeutics”, University of South Florida College of Medicine, Tampa, Florida, 33612.
Zurier, Prospects for cannabinoids as anti-inflammatory agents, Department of Medicine, Division of Rheumatology, University of Massachusetts Medical School, Worcester, USA. Robber
Kagan, et al., CONSUMERS' PERCEPTIONS OF HEMP PRODUCTS FOR ANIMALS, Department of Clinical Sciences, the College of Veterinary Medicine and Biomedical Sciences, Colorado State University, Fort Collins, Colo. 80526.
Mbvundula, et al., Cannabinoids in pain and inflammation, Inflammopharmacology 2004;12 (2):99-114.
Inke Paetau-Robinson, PhD, et al Long-Term Feeding of DL-a Lipoic Acid to Dogs Is Safe, Intern J Appl Res Vet Med. Vol. 11, No. 2, 2013
Kogan, et al., US Veterinarians' Knowledge, Experience, and Perception Regarding the Use of Cannabidiol for Canine Medical Conditions, Frontiers of Veterinary Science, January 2019—Volume 5—Article 338.
Nutrient Content Claims; Alpha-Linolenic Acid, Eicosapentaenoic Acid, and Docosahexaenoic Acid Omega-3 Fatty Acids; Guidance for Industry Small Entity Compliance Guide, Office of Nutrition and Food Labeling Nutrition Programs Staff, HFS-830 Center for Food Safety and Applied Nutrition Food and Drug Administration 5001 Campus Drive College Park, MD 20740.
Kwang-GeunLee, et al, Antioxidant property of aroma extract isolated from clove buds [Syzygium aromaticum (L.) Men. et Perry]—Food Chemistry, Volume 74, Issue 4, Sep. 2001, Pages 443-447
Omga-3 Fatty Acids, Fact Sheet for Health Professionals, The National Institute of Health, Nov. 21, 2018
Michael Backes, Andrew Weil, MD, Cannabis Pharmacy: The Practical Guide to Medical Marijuana.

What I claim:

1. A dietary supplement composition comprising essentially of:
(a) about 0.1 percent to 50.0 percent by weight of a Cannabis sativa extract;
(b) 10 percent to 99.9 percent by weight of edible oil comprising of less than 15 percent by weight of saturated fats and greater than 44 percent by weight of polyunsaturated fats;
(c) 0 to 50 percent by weight of a non-ionic surface active agent;
(d) 0 to 10 percent by weight of a vitamin;
(e) 0 to 5 percent by weight of an antioxidant;
(f) 0 to 5 percent by weight of a flavoring agent; and
(g) 0 to 2 percent by weight of a preservative.

2. The composition of claim 1, wherein (a) is selected from Cannabis sativa extracts comprised of one or a combination of cannabinoid compounds.

3. The cannabinoid compounds of Cannabis sativa extracts of claim 2, comprise of: Cannabidiol also know as CBD; Cannabinol also known as CBN;
Cannabigerol also known as CBG; Cannabidiolic acid also known as CBD-A;
Cannabichromene also known as CBC; Delta-9-tetrahydrocannabinol also known as Δ-9-THC; Delta-9-tetrahydrocannabinol Acid also known as Δ-9-THC-A;
Delta-8-tetrahydrocannabinol also known as Δ-8-THC; and
Tetrahydrocannabivarin also known as THC-V.

4. The preferred cannabinoid compound of claim 3, is Cannabidiol, also know as CBD.

5. The preferred level of use of the Cannabidiol also know as CBD of claim 4, is from 0.5 percent to about 15 percent.

6. The most preferred level of use of cannabidiol of claim 4, is from 1.5 percent to about 7.0 percent.

7. The composition of claim 1, wherein (b) is selected from naturally derived, edible oils that comprise of: Borage Seed Oil, Chia Seed Oil, Cranberry Seed Oil, Elderberry Seed Oil, Flax Seed Oil, Grape Seed Oil, Hemp Seed Oil, Maracuja Seed Oil, Perilla Seed Oil, Poppy Seed Oil, Safflower Seed Oil, Soya Bean Oil and Sunflower Seed Oil.

8. The preferred Edible Oils of claim 7, are: Borage Seed Oil, Chia Seed Oil, Cranberry Seed Oil, Elderberry Seed Oil, Grape Seed Oil, Hemp Seed Oil, Safflower Seed Oil, and Sunflower Seed Oil.

9. The composition of claim 1, wherein (c) is selected from surface active agents that comprise of, but not limited to, of one or more of FDA approved food grade non-ionic surface active agents having a hydrophilic/lipophilic balance (HLB) greater than 9 and less than 25.

10. The selected non-ionic surface active agents of claim 9, are further described as: Tween 85 (Polyoxyethylene sorbitan trioleate), Tween 81 (Polyoxyethylene sorbitan monooleate), Tween 80 (Polyoxyethylene sorbitan monooleate), Tween 65 (Polyoxyethylene sorbitan tristearate), Tween 61 (Polyoxyethylene sorbitan monostearate), Tween 60 (Polyoxyethylene sorbitan monostearate),
Tween 40 (Polyoxyethylene sorbitan monopalmitate), Tween 21 (Polyoxyethylene sorbitan monolaurate), Tween 20 (Polyoxyethylene) sorbitan monolaurate; non-ionic surfactants in the form of ethers such as: Brij O20 (Polyoxyethylene (20) oleyl ether), Brij L4 (Polyoxyethylene (4) lauryl ether), Brij S100 (Polyoxyethylene (100) stearyl ether); and non-ionic surfactants in the form Copolymers of polyethylene glycols and polypropylene glycols such as:
Pluronic F68 and F127.

11. The preferred non-ionic surfactant active agents of claim 10, are: Tween 20, Tween 21, Tween 80 and Tween 81 and are further described as: Polyoxyethylene sorbitan monolaurates.

12. The composition of claim 1, wherein (d) is selected from vitamins that comprise of, but not limited to, one or more of: Vitamin A (retinol), Vitamin C as Ascorbyl Palmitate, Vitamin D₃, Vitamin E including tocotrienol and tocopherol and its esters.

13. The antioxidants comprise of but not limited to: one or a combination of the following: Alpha Lipoic Acid, Carotenoid terpenoids such as: Alpha-carotene Astaxanthin, Beta-carotene, Canthaxanthin, Cryptoxanthin, Lutein, Lycopene, and Zeaxanthin; Flavonoids such as: Flavones such as: Apigenin, Luteolin, and Tangeritin; Flavonols such as: Isorhamnetin, Kaempferol, Myricetin, and Quercetin; Flavanones such as: Eriodictyol, Hesperetin, and Naringenin; Flavanols and their polymers such as: Catechin, gallocatechin and their corresponding gallate esters, Epicatechin, epigallocatechin and their corresponding gallate esters, and Theaflavin and its gallate esters; Isoflavone phytoestrogens such as: Daidzein, Genistein, and Glycitein; Stilbenoids such as: Resveratrol, and Pterostilbene, Anthocyanins: Cyanidin, Delphinidin, Malvidin, Pelargonidin, Peonidin, and Petunidin; Phenolics such as: Curcumin, Flavonolignans, Xanthones, and Eugenol.

14. The composition of claim 1, wherein (f) is Sesame Seed Oil for flavoring domestic animal compositions and other compatible flavoring agents known to those in the arts.

15. The composition of claim 1, wherein (g) is selected from preservatives that comprise of one or a combination of the following: Butylated Hydroxy Toluene (BHT), and Butylated Hydroxy Anisole (BHA).

16. A method of increasing the bioavailability of Cannabis sativa extracts from an orally delivered composition where said method is in a liquid dosage form to be incorporated into a dietary supplement or pharmaceutical dosage comprising:
(a) about 0.1 percent to 50.0 percent by weight of a Cannabis sativa extract;
(b) 10 percent to 99.9 percent by weight of edible oil comprising of less than 15 percent of saturated fats and greater than 44 percent of polyunsaturated fats;
(c) 0 to 50 percent by weight of a non-ionic surface active agent;
(d) 0 to 10 percent by weight of a vitamin;
(e) 0 to 5 percent by weight of an antioxidant;
(f) 0 to 5 percent by weight of a flavoring agent; and
(g) 0 to 2 percent by weight of a preservative.
